(19) United States
(12) Patent Application Publication
(10) Pub. No.: US 2008/0221669 A1
(43) Pub. Date: Sep. 11, 2008

(54) Endovenous Stent and Venous Neovalvular Endobioprosthesis

(75) Inventors: Sante Camilli, Roma (IT); Daniele Camilli, Via Casole d'Elsa (IT)

Correspondence Address: YOUNG & THOMPSON, 209 Madison Street, Suite 500, ALEXANDRIA, VA, 22314, US

(73) Assignee: Sango S.A.S di Cattani Rita e C., Roma (IT)

(21) Appl. No.: 11/996,980

(22) PCT Filed: Jul. 27, 2005
(86) PCT No.: PCT/IT05/00450

§ 371 Date: Jan. 28, 2008

(51) Int. Cl. A61F 2/06

(52) U.S. Cl. 623/1.24

(57) ABSTRACT

Endovenous stent for restoring the function of an incompetent venous valve having slackened cusps. The stent includes two parallel struts; two terminal elastic elements, connecting the struts at their extremities and compressing them apart, and make them, once the stent implanted into the vein to be cured, to dilate the intercommissural diameter of the valve to be cured, taking up the slackening of the incompetent cusps; and such endovenous stent, further dressed with a venous valvulated segment reversed inside-out, in which the cusps perform their valvular function by moving themselves from inside out, so constituting a venous neovalvular endobioprosthesis for curing an incompetent vein by implantation into it, in case of damage or aplasia of the venous valve.

### TECHNICAL FIELD

This invention relates to the field of venous valve restoration in venous surgery for curing valve incompetence, which leads to chronic venous insufficiency (CVI).

More specifically, this invention relates to a novel endovenous stent for venous valve restoration, implantable by percutaneous route.

This invention also relates to an endovenous neovalvular endobioprosthesis fabricated starting from said novel endovenous stent.

### BACKGROUND ART

Relevant prior art can be classified in three main conceptual groups.

The first one generally relates to venous valve endoprostheses mimicking a natural valve.

WO2004/016200, D. PAVCNIK et al.; US Patent Appln. Pub. no. US2004/0193253, THORPE et al.; CA2 441 999, Cordis Corp., inventors DUERIG AND MELZER; WO03/071990, FISCHER AND VOGEL; US2002177894, KICK AND ACOSTA; U.S. Pat. No. 6,716,241, J. G. WILDER AND A. TESAR, disclose implantable venous devices which constitute substitutes mimicking natural valves including leaflets which close themselves from the venous wall towards its inside to stop the reflux of blood.

WO03/070124, OSSE and THORPE, discloses a replacement venous valve having a deployable configuration of struts and membranes which open under blood reflux, stopping it whilst remain closed under normal orthograde blood flow.

U.S. Pat. No. 6,602,286, STRECKER, discloses an implantable valve including a leaflet and a base that is attachable to a blood vessel wall using a mechanical connector, such as a screw. The leaflet is a monoleaflet shaped as a saddle which arranges itself in a closing position transversely oblique and rises for the opening of the vessel letting orthograde blood flow to pass.

Other valves are disclosed in U.S. Pat. No. 6,319,281, R. PATEL KUMAR; U.S. Pat. No. 6,315,793, BOKROS et al.; U.S. Pat. No. 6,299,637, S. M. SHAOLIAN and G. VON HOFFMANN; all these ones disclose totally prosthetic valves, intended to be substitutes of natural valves.

However, no venous valvular endoprosthesis of such first group has actually correctly been able to perform its function, owing to the fundamental fact that they all promote thrombosis too much and then the mechanical block thereof. In fact, none of them is actually on the market nor actually used in surgery.

The second conceptual group includes the endovenous stents which operate on the principle of modifying the cross section of the vein.

J. F. VAN CLEEF, WO 9301764, teaches an endovenous stent implantable by percutaneous route based on the concept that a vein can be laterally forced towards outside modifying its diameters and flattening it like a ribbon with the object of making a routine pharmacological sclerotherapy more effective and durable, without the frequently occurring recanalization. The endoprosthesis is implantable in any point of the conduit of the vein to be cured, typically in proximity to the sapheno-femoral junction. The operating principle of this endoprosthesis is to block an induced thrombus to cure the venous insufficiency by excluding the conduit of the affected vein out of the blood circulation.

The disadvantage of VAN CLEEF'S curing principle is that it involves the functional destruction of the vein itself with a consequent development of the collateral centripetal circulation, which is very likely a cofactor of the varicose vein recurrence.

U.S. Pat. No. 6,527,800, J. F. MCGUCKIN and R. T. BRIGANTI, discloses an endovenous stent and method for valve leaflet apposition, wherein such curing is effected by means of the reduction of the diameter of the vein segment adjacent and immediately caudad relative to the valvular bulb by traction from the inside of the vein onto the walls thereof grasped by hooks and by a modification of the predetermined shape of the device by a thermal memory treatment. The envisaged endovenous stent operates a circumferential reduction of the cross section of the vein, immmediately caudad relative to the valve bulb, so inducing an indirect angioplasty of the valve bulb.

MCGUCKIN, however, has the disadvantage that, ultimately, it aims at only roughly promoting the contact between the slackened valvular cusps by restricting the space where they move, operating a circumferential retraction of the wall of the vein, which does not modify the slackening of the leaflets, and renders the result uncertain and approximate; in fact, it acts on the caudal segment of the bulb of the valve instead of on the cranial segment, which is the critical one for the tension and position control of the leaflets, as it is there that the attachement lines as well as the free edges of the leaflets converge.

The third conceptual group includes documents which envisage the concept of applying the principle of modifying the cross section of the vein, ovalizing it, to restore valve function, by a force applied onto the external wall of a valve bulb of a vein.

ZUKOWSKI, WO97/40755, discloses a stent based on the discovery that an external force applied onto a vein, at the level of the valve bulb where a slackened and incompetent valve of such vein is present, flattens the vein with the aim to extend the cusps of the valve laterally, taking up their slackening and bringing them into apposition, restoring the competence of the vein. So ZUKOWSKI'S device features a support for applying an external, compressive force to an incompetent vein.

Problems are associated with ZUKOWSKI'S device. The compression from outside actually does not ensure a contemporaneous increase of the intercommissural diameter per se. It is a disadvantage of ZUKOWSKI'S device that its compressive action actually is not reliable as regards its corrective action.

S. CAMILLI, unpublished European Patent Application 04.425074.4, discloses an external support for restoring competence to venous valves by traction of their intercommissural walls, which applies an external traction force directly on the intercommissural walls, with the aim to dilate the intercommissural diameter so tightening it and taking up the slackening of the cusps.

The third group has the disadvantage of requiring a surgical incision for their application.

### DISCLOSURE OF THE INVENTION

The object of this invention is to provide an endoprosthesis which operates on the basis of a principle of curing CVI, and especially varicose veins, by venous valve restoration both to obtain the backward flow blocking and to maintain the orthograde blood flow, which actually recovers valve function, which is minimally invasive and implantable by percutaneous route.

Such an object, according to the teaching of this invention, is reached by means of the novel concept of tightening the valvular cusps—which have become longer or asymmetrical owing to the pathologic process—by the application of an endovenous pushing, internal force onto the walls of the vein corresponding to the valvular commissures, i.e. onto the point where the valvular cusps attach themselves to the wall of the vein to constitute the free or mobile edge of the valve cusps. The inventive endoprosthesis operates by dilating a vein, at the level of the valve bulb, along its intercommissural diameter to reabsorb the excessive length of the slackened cusps that cause the venous incompetence.

The inventive endovenous stent ultimately has a characteristic of performing a restorative valve plastics by dilating the intercommissural diameter of the vein, so ovalizing the cross section of the native valve bulb itself.

For these characteristics the inventive endovenous stent turns out to be novel relative to prior art set-forth above.

Particularly, no endoprosthesis of the first prior art group has anyone of the inventive characteristics.

As regards the second group, the inventive endovenous stent aims at keeping a vessel wide-open to maintain forward flow, and so it differs from VANCLEEF, which aims at permanently occluding the vein by shaping it into a ribbon.

It equally differs from MCGOUKIN, which operates by traction instead of by pushing on the walls, circumferentially reducing the vein cross section instead of ovalizing it, by indirect valve plastics instead of the direct valve bulb plastics performed with the present invention.

As regards the third group, the inventive endovenous stent applies the ovalizing force from inside out, whilst prior art devices apply its ovalizing force from outside in.

The stent can be fabricated by a top-down process envisaging a laser-cutting of a metal tube, made up for instance by Nitinol® alloy, followed by a thermal treatment and an electro-polishing treatment.

It is an advantage of the inventive endovenous stent that it can be implanted by percutaneous route by utilizing a suitable delivery system by virtue of the fact that it is collapsible, so it can be loaded into said delivery system and thereafter deployed at a proper implantation site, e.g. under fluoroscopic or duplex-scanning and/or endoscopic monitoring.

Under another aspect, this invention relates to a neovalve obtained by the above referred inventive endovenous stent.

Clinical cases are known wherein the native venous valves are asymmetrical, thickened, ruptured or otherwise damaged, as well as cases with venous valve aplasia. In these cases the inventive endovenous stent cannot work as a such. There are also cases on which the endovenous stent positioning was envisaged as a primary option, but the exactness of positioning was not reached or cannot be reached for some reasons.

In these cases, the inventive endovenous stent may equally work in combination with a venous valvulated segment, turned inside-out and secured and tightened by the stent, obtaining a neovalve, i.e. a newly constructed valve, constituting a venous neovalvular endobioprosthesis.

The venous valvulated segment can be an autologous segment or can be harvested from a donor, human or animal subject, or human cadaver, according to rules relevant to organ explantation.

The neovalve is inserted into the host vein as a septum which divides the lumen of the vein into two half-lumina, as opposed to prior art.

It is a characteristic of such venous neovalvular endobioprosthesis that its closing is actuated by an opening motion of the cusps thereof from inside out as opposite to native valves, which open from outside in.

In prior art related thereto, document CA2360175, J. GOMEZ-JORGE et al., discloses a venous neovalvular endobioprosthesis fabricated by using a valvulated vein segment supported by a self-expandable stent. The stent is intended to be arranged externally on a vein segment. The valvulated vein segment is not turned inside-out, so that the valvular leaflets or cusps work in their normal position, opening from outside in.

J. G. WILDER et al., U.S. Pat. No. 6,716,241, Venous valve and graft combination, discloses a synthetic trilobed venous valve. This one exerts no radial force at the valve site, and operates its function with a movement of the cusps from outside in; differently from the instant neovalve which operates by radial forces applied inside-out modifying the circular cross section into an ovalized one, and with the cusps moving inside-out for the closing.

N. M. WILSON et al., In Situ Venous Valve Construction (Br. J. Surg. 1991; 78:595), discloses an autologous neovalve constructed by a surgical technique envisaging the intussusception of a caudal vein segment into a proximal or cranial one, overlying it. This one, however, has a drawback in that it excessively reduces vasal lumen, can promote thrombosis and finally needs a particular skillness of the surgeon.

RU2160057, A. A. DJUZHIKOV et al., WO2004/037128, W. J. DRASLER and M. L. JENSON, disclose a venous valve apparatus and method applying WILSON'S teaching above. They suffer from the same drawbacks.

US Patent Appln. Publication No. 2002/0099439, R. S. SCHWARTZ and R. A. VAN TASSEL, disclose a venous valvuloplasty device preferably constructed of a material capable of promoting cellular ingrowth such that, eventually, native biologic tissue completely covers the device. The device is sized and arranged to mimic the valve it is replacing or repairing.

EP1172120, L. DE SUNNANVAEDER and A. BADER, discloses an individual venous valve prosthesis constructed from a matrix utilizing a biologic tissue treated in the laboratory so that it can be covered by native endothelial cells, provided with at least one leaflet.

It is an advantage of the inventive venous neovalvular endobioprosthesis that it may be used in cases of damaged or absent native valve, it may be used—as a second option—in the case where the endovenous stent cannot reach an exact positioning on the intercommissural diameter of the native valve for some reason, it may be positioned at any level into the venous conduit without respect to a pre-existing venous valve.

### BRIEF DESCRIPTION OF THE DRAWINGS

This invention will be fully understood based on the following disclosure, only given as a matter of example and not of restriction, reference being made to the annexed drawings wherein:

FIG. 1A depicts the front-rear view of a first embodiment of the inventive endovenous stent, inserted into a vein, in correspondence with an incompetent native venous valve, tightening its slackened free edge;

FIG. 1B depicts the side-lateral view of such first embodiment, inserted into a vein;

FIG. 1C depicts the axial section view of a vein with such inventive endovenous stent of the first embodiment implanted thereinto, in its tightened and closed position, according to line A-A passing through the apices of the valve commissures of one the cusps;

FIG. 2A depicts the front-rear view of a second embodiment of the inventive endovenous stent;

FIG. 2B depicts the side-lateral view of such second embodiment;

FIG. 2C depicts the axial view of a vein with such second embodiment of the inventive endovenous stent implanted thereinto, in its tightened and closed position;

FIG. 3A depicts the front-rear view of a venous neovalvular endobioprosthesis as obtained from the above-referred inventive endovenous stent according to the second embodiment of FIG, 2A to 2C, with the addition of a valvulated venous segment turned inside-out;

FIG. 3B depicts the side-lateral view of said venous neovalvular endobioprosthesis with its valve cusps in open position, and

FIG. 3C depicts the axial view of a vein with the venous neovalvular endobioprosthesis of FIG. 3A and 3B implanted thereinto, in its semi-closed position.

### BEST MODE OF CARRYING OUT THE INVENTION

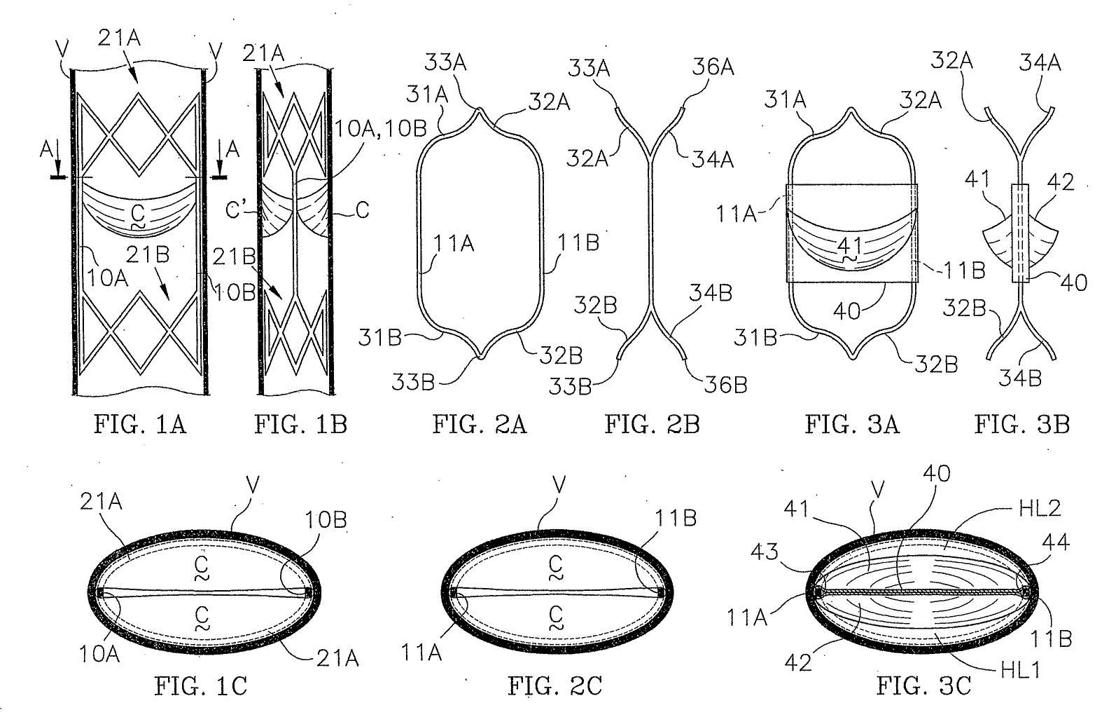

Referring to FIG. 1A and 1B, an inventive endovenous stent includes a pair of coplanar parallel struts 10A and 10B, which can have the same length. These ones are connected by their extremities by two terminal elastic elements in the shape of elliptic “crowns” 21A and 21B, in the same material as the struts, built with the structure of self-expandable stents (well known in the art). Such terminal elements are shaped for a mutual conforming between them and the wall of a vein. The two coplanar parallel struts 10A and 10B, once the stent introduced in vein V, perform the function of dilating the intercommissural diameter of a vein up to the required length, under the compressive force of terminal elements 21A, 21B, so ovalizing the vein as decided by the surgeon. Terminal elastic elements 21A and 21B perform the function of anchoring the stent to the wall of a vein. It is also a function of the latter to keep the vein lumen wide-open in case of an excessive flattening of the vein induced by an oversizing of the stent.

The sizing and the performance of the stent are substantially determined by the terminal elements.

Referring to FIG. 1C, the inventive stent, once implanted into a vein V, under the force of terminal crowns 21A and 21B, struts 10A and 10B urge the venous wall, so that the latter takes an ovalized cross-section from the normal circular one, intercommissural diameter of vein V becoming greater, whilst the diameter orthogonal thereto becoming smaller. In this way, the slackening of the valve cusps C is taken up so that they are able again to coapt with each other to perform their valving function, and the object of the invention is reached.

Referring to FIG. 2A to 2C, an alternate embodiment of the inventive stent—instead of the mentioned “crown” terminal elements—includes two “episcopal mitre” terminal elements. Upper episcopal mitre terminal element includes two specular elements respectively made up of two S-shaped arcs 31A, 32A; 33A 34A. In each specular element S-shaped arcs are connected by an extremity to a respective strut and together by the other extremity at an apex 35A, 36A. The arcs are bent in space, in such a way as to symmetrically protrude outerly of the plane of the struts, by opposite parts thereof, determining a concavity as seen in side view as in FIG. 2B.

The two disclosed embodiments constitute a means for restoring functionality to an existing native valve.

For the case of dilated valve bulbs and/or slackened cusps, the inventive endovenous stent, according to one of the disclosed embodiments, is released into the site of the valve bulb to be cured. Under angioscopic and/or fluoroscopic and/or duplex scanning monitoring, the stent has to be delivered exactly at the bulb valvular site, so that the central portion of the stent, of appropriate size, is pushing longitudinally on the intercommissural walls. It is foreseen that the intercommissural diameter is to be increased of about 20-30% so as to take up the slackening of the incompetent valve cusps.

Referring to FIG. 3A to 3C, a neovalve is shown, obtained from the disclosed endovenous stent, which constitutes a venous neovalvular endobioprosthesis, which is fit for the case of valve hypoplasia or agenesia or in the case of damaged cusps, where a valve restoration cannot be put into practice. A valvulated vein segment 40 is harvested e.g. from the lower or upper limb of the patient and subjected to microsurgical excision of outermost, thicker layers of the vein. Said segment is turned inside-out. At this point, the inventive endovenous stent—as in anyone of the embodiments disclosed above—is introduced into the valvulated vein segment 40, flattening the latter into a ribbon shape. The cusps C1, C2, of the reversed vein segment lie on the outside of the latter, opposite to each other.

The venous neovalvular endobioprosthesis, so obtained, is to be fitted to a host vein V to be cured by implanting the assembly into it. The valvulated venous segment 40, by the redoubled thickness of its collapsed walls, divides the lumen of the vein as a septum into two half-lumina HL1, HL2.

In FIG. 3B the two opposite valve cusps C can be seen in the condition of open cusps, which is the closing condition of the venous neovalvular endobioprosthesis.

Referring to FIG. 3C, a venous neovalvular endobioprosthesis is shown once implanted into a vein, where V is the host vein, 11A and 11B are the cross sections of the two stent struts, 40 is the ribbon-like shaped harvested vein segment turned inside-out, 41 and 42 are the two leaflets in a semi-open condition. As in the case of the above described embodiment, the inventive venous neovalvular endobioprosthesis makes vein V to take an ovalized cross section from the normal circular one, so that the intercommissural diameter of the vein becomes greater, whilst the diameter orthogonal thereto becomes smaller. In this way, leaflets 41 and 42 have to cover a smaller span to coapt with the walls of native vein V.

The length of the struts can be of 8 to 12 mm, the length of the terminal elements can be of 4 to 8 mm; the rest maximum diameter is about 8 to 20 mm, the minimum rest diameter can be 4 to 12 mm; the strut caliber can be 0.20 to 0.30 mm; being it understood that all the measures depend on the size of the vein to be treated.

### INDUSTRIAL APPLICABILITY

The endovenous stent can be fabricated in a biocompatible plastic or a metallic material, e.g. AISI 316L or other medical grade steel or alloy, by any known technique. E.g. the stent can be fabricated with a laser-cut Nitinol® tubelet subjected to electropolishing to smooth all acuminated parts.

The venous valvular endobioprosthesis can be fabricated on the bench, using an autologous valvulated venous segment; it can be preserved by anyone of the methods already known, e.g. a glutaraldehyde fixation treatment, or a cryopreservation treatment, so that it can be implanted subsequently as a custom-made implantable medical device. It may overcome an impossibility of the surgeon to realize the endobioprosthesis in the same surgical session.

The venous valvular endobioprosthesis can be fabricated on the bench, using an homologous—typically from a human donor or a human cadaver—or an heterologous valvulated venous segment or xenograft—from selected animals—or even an allograft; it can be preserved and sterilized, as above, for commerce so that it can be implanted subsequently as a custom-made, as well as a free-marketable implantable medical device.

1-4. (canceled)

5. Endovenous stent for restoring the function of an incompetent venous valve in a vein to be cured, having an intercoinmissural diameter and including incompetent slackened cusps; which endovenous stent includes in combination two—and only two—parallel elastically flexible side struts; two terminal elastically flexible elements, which connect said struts at the extremities thereof and compress them apart from each other, so as to space them apart, and which are so shaped as to conform to the internal wall of a vein and to oval the cross-section thereof, keeping the vein lumen wide-open, once the endovenous stent implanted thereinto, so as to dilate the intercommissural diameter of said incompetent venous valve, taking up the slackening of said incompetent slackened cusps thereof once the endovenous stent implanted in said vein to be cured, in correspondence with the incompetent venous valve.

6. Endovenous stent as in claim 5, wherein said terminal elastically flexible elements are crown-(21A, 21B) or episcopal-mitre-(31A, 32A, 33A, 34A, 36A; 31B, 32B, 33B, 34B, 36B) shaped elements.

7. Endovenous stent as in claim 5, having a structure which can be made to collapse to a reduced diameter, so as to constitute a self-expandable stent, implantable by percutaneous route, by catheter.

8. Endovenous stent as in claim 5, further equipped with a human or animal venous valvulated segment, having two cusps (41, 42), so reversed that the inner wall thereof is made the outer wall thereof and vice-versa; the endovenous stent being inserted in said reversed venous valvulated segment; said reversed venous valvulated segment being ribbon-like (40) flattened and having said two cusps (41, 42) thereof opposite to each other; the extremities of said reversed venous valvulated segment being secured to each other by suturing or stapling or gluing; the so equipped endovenous stent so constituting a venous neovalvular endobioprosthesis for curing an incompetent vein, by implantation into it at any level of a venous conduit, including the level of an incompetent venous valve thereof.
